(19) United States
(12) Patent Application Publication
(10) Pub. No.: US 2016/0101244 A1
(43) Pub. Date: Apr. 14, 2016

(54) INHALER FOR A METERED DOSE AEROSOL

(71) Applicant: CHV PHARMA GMBH & CO. KG, Wiesbaden (DE)

(72) Inventor: Björn O. Sörensen, Wiesbaden (DE)

(21) Appl. No.: 14/787,435

(22) PCT Filed: May 2, 2014
(86) PCT No.: PCT/EP2014/059008

§ 371 Date: Oct. 27, 2015

(30) Foreign Application Priority Data
May 3, 2013 (DE) 10 2013 104 549.9

(51) Int. Cl. A61M 11/00; A61M 15/00

(52) U.S. Cl. CPC A61M 11/003; A61M 15/009

(57) ABSTRACT

An inhaler for delivering a metered dose aerosol, in which a permeable sheet is positioned directly in front of a narrow channel.

### FIELD OF THE INVENTION

The invention relates to an inhaler for a metered dose aerosol comprising a housing for receiving a drug container.

### BACKGROUND OF THE INVENTION

Inhalers for metered dose aerosols are known. They typically comprise a housing for receiving a drug container. Such a drug container contains a propellant and the drug. Furthermore, the drug container comprises a valve, and upon actuation of the valve a defined amount of the drug is delivered.

In the most common inhalers the valve is actuated by the user pressing on the drug container inserted into the housing of the inhaler. Such an inhaler is disclosed in DE 601 32 666 T2.

The housing typically comprises an atomizing nozzle through which an aerosol is emitted into a mouthpiece of the housing, which aerosol can be inhaled by the user.

A problem with known inhalers is that they emit a relatively short puff with high particle velocity. This requires good coordination in actuating the puff and inhaling the aerosol cloud. Usually, part of the drug is not inhaled.

Various approaches are known to mitigate this problem. In particular, relatively large-volume attachable containers are known from practice, in which the aerosol cloud is expected to accumulate so that the user can inhale it. However, these containers are impractical and have the drawback that depending on the aerosol used, more or less aerosol is deposited on the container wall.

Patent document U.S. Pat. No. 4,706,663 A proposes to place an extension on the inhaler, which is closed with a screen. Particle velocity is intended to be reduced in this way. However, in the past such approaches have led to strong fluctuations in the delivered dose (total emitted dose) due to the fact that particles are deposited.

A general problem with many inhalers known from practice is a high standard deviation in the delivered amount of the drug. It is assumed that deposition of the drug in the area of the nozzle also occurs in conventional inhalers that comprise a spray nozzle, which leads to fluctuations in the amount of the drug delivered.

### OBJECT OF THE INVENTION

Therefore, the invention is based on the object to mitigate the aforementioned drawbacks of the prior art.

In particular, an object of the invention is to provide a simply constructed inhaler in which particle velocity is reduced compared to known inhalers and/or in which the accuracy of the delivered amount of drug is improved.

Furthermore, the invention relates to an inhaler with improved handling.

### SUMMARY OF THE INVENTION

The object of the invention is already achieved by an inhaler for delivering a metered dose aerosol according to the illustrative embodiment of the present invention.

The invention relates to an inhaler for a metered dose aerosol, that is a device by means of which a liquid, in particular a suspension, which includes an active substance may be delivered as an aerosol cloud. The active substance is usually a drug. However, it shall not be excluded within the sense of the invention that the inhaler may also be used for delivering other substances, for example for delivering stimulants.

The inhaler comprises a seat for the outlet of a drug container. Such a seat is usually formed as a kind of bore into which a tubular outlet of the drug container inserted into the housing can be introduced.

Furthermore, the inhaler comprises a channel extending from the seat for the outlet of the drug container to an atomizer outlet.

The atomizer outlet refers to a component at which the aerosol enters the mouthpiece of the inhaler.

The atomizer outlet comprises a sheet that is permeable to the aerosol. In particular, the atomizer outlet comprises a nonwoven fabric, a mesh, or a woven fabric. The channel provided between the outlet of the drug container and the atomizer outlet and leading to the permeable sheet is constricted in sections thereof.

Thus, the channel comprises a constriction. This constriction in particular has a diameter between 0.1 and 1 mm, preferably between 0.3 and 0.7 mm.

The constriction which is close to the permeable sheet leads to a considerable increase in velocity of the liquid in the area of the constriction.

The inventor assumes that this prevents larger amounts of particles from depositing on the permeable sheet or on the walls of the channel.

Rather, a decrease in velocity of the particles in the aerosol cloud could be achieved, which in particular results in a larger and better inhalable aerosol cloud. However, at the same time the standard deviation of the amount of delivered drug was even reduced compared to inhalers known from practice.

It has been found that when the inhaler is used for a suspension, a diameter of the constriction from 0.4 to 0.6 mm, in particular 0.5 mm, is optimal.

When the inhaler is used for a solution, a slightly smaller diameter is optimal, in particular from 0.2 to 0.3 mm, most preferably 0.25 mm. Advantageously, the constriction is formed by two converging and tapering channel sections which are arranged at an angle relative to each other.

For a particularly efficient delivery, an angle between 100° and 130°, preferably between 110° and 120° has been found particularly suitable.

Preferably, the angle between the two channel sections is equivalent to the angle between the main direction of extension of the mouthpiece and a rotational axis of symmetry of the drug container.

In this case, the constriction preferably extends approximately with the same inclination as a central axis of the mouthpiece.

The permeable sheet preferably has a mean pore diameter between 10 and 100 μm, particularly preferably between 30 and 50 μm.

The diameter of the opening of the atomizer outlet, i.e. the diameter of the permeable sheet is preferably between 0.5 and 3 mm, particularly preferably between 1 and 2 mm.

When using a membrane, in particular in form of a nonwoven fabric, and when using the inhaler for a solution, mean pore diameters from 50 to 70 μm, in particular of about 60 μm have been found ideal.

The permeable sheet preferably has an open surface area between 10 and 60%, particularly preferably between 25 and 45%.

The invention further relates to an inhaler for a metered dose aerosol in particular as described above, which comprises a seat for the outlet of a drug container and a channel that extends from the seat to an atomizer outlet.

The atomizer outlet comprises the aforementioned permeable sheet which closes the channel by means of which the outlet of the drug container is connected.

This channel has a length of less than 8 mm, preferably less than 5 mm. It has been found that with a particularly short and preferably moreover fairly thin channel, uniform drug delivery is achieved.

Presumably this effect is due to the fact that upon actuation of the inhaler, there is a pressure buildup and an acceleration of the aerosol particles being formed in the channel.

In this way, in particular in conjunction with a diameter of the permeable sheet of less than 3 mm, a deposition of drug particles is largely avoided.

It has been found that the inhaler according to the invention is in particular also suitable for suspensions. In a preferred embodiment of the invention, the channel has a volume of less than 10 mm³, preferably less than 5 mm³, and most preferably less than 3 mm³.

The invention further relates to an inhaler for a metered dose aerosol, in particular as described above, which comprises a container housing for receiving a drug container and an atomizer outlet which opens into a mouthpiece.

According to the invention, the mouthpiece has an extension at an end opposite the opening for inhaling the aerosol.

In particular, the mouthpiece protrudes by at least 10 mm at a rear end opposite the opening.

The extension facilitates better handling since the inhaler is better balanced in the hand of the user in this manner.

Furthermore, it is conceivable to use the volume created by the extension for integrating further components, in particular for integrating a counting device.

### DETAILED DESCRIPTION

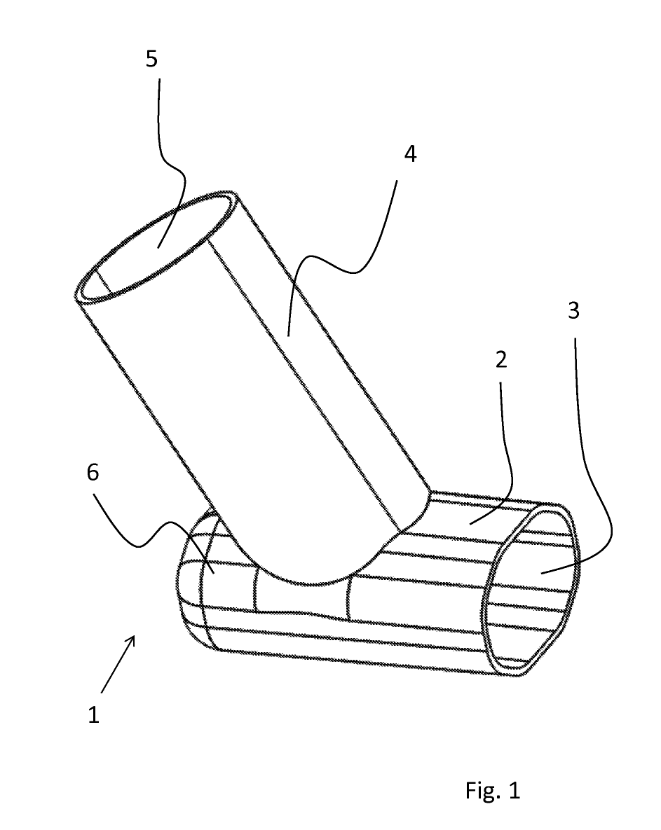

The subject matter of the invention will be described in more detail below by way of schematically illustrated exemplary embodiments and with reference to the drawings of FIG. 1 to FIG. 7.

FIG. 1 is a schematic perspective view of an exemplary embodiment of the housing of an inhaler 1. In this view, the drug container is not inserted.

Inhaler 1 comprises a mouthpiece 2 having an opening 3 through which the user inhales the emitted aerosol. Mouthpiece 2 is arranged at an angle differing from 90°, in this exemplary embodiment at an angle from 110° to 120° relative to a container housing 4, which has a tubular shape with a circular cross section at least on the inside thereof, and which has an opening 5 at the top for inserting the drug container (not shown).

Mouthpiece 2 furthermore comprises an extension 6 at the rear end, i.e. opposite to opening 3, enabling better balancing when using the inhaler.

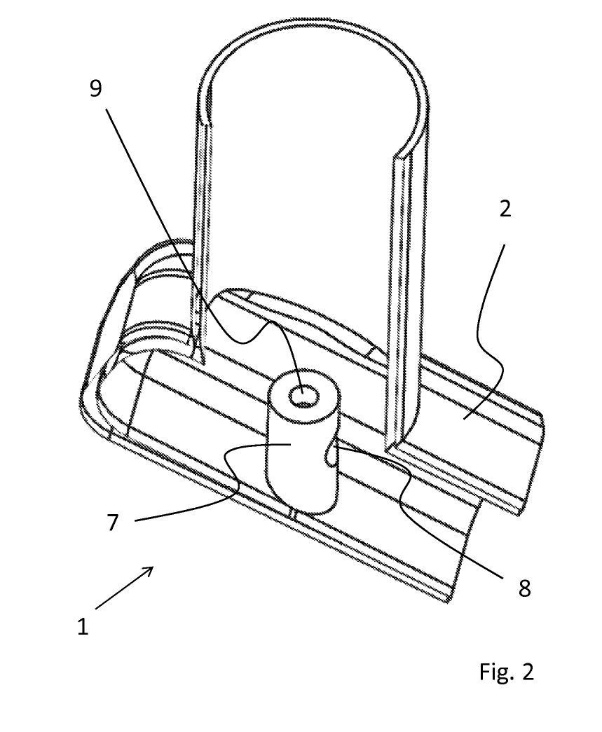

FIG. 2 shows a perspective cutaway view of the inhaler 1 shown in FIG. 1.

As can be seen in this view, a seat 7 is protruding into mouthpiece 2, which seat has a channel 9 into which the outlet of a drug container (not shown) can be inserted.

Furthermore, an atomizer outlet 8 can be seen, from which the aerosol cloud is emitted upon actuation of the inhaler.

The configuration of seat 7 as a socket which in this exemplary embodiment has an essentially circular cylindrical shape has the advantage that a large volume remains in mouthpiece 2.

This volume which also exists at an end opposite the mouthpiece opening may be used to integrate additional components, in particular a counting device, for example.

The illustrated embodiment allows for a particularly easy manufacturing of inhaler 1, for example as a plastic injection molded part in which seat 7 is an integral part of the housing.

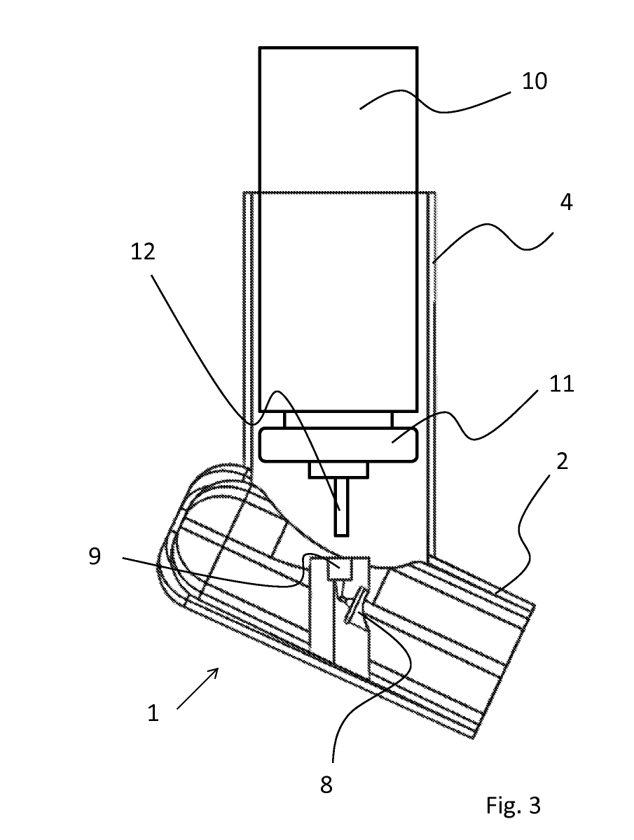

FIG. 3 is a schematic sectional view of inhaler 1, and in this view a drug container 10 is shown being inserted into container housing 4.

Drug container 10 has a collar 11 and a tubular outlet 12.

Tubular outlet 12 is inserted into channel 9.

If, now, drug container 10 is further pressed down, outlet 12 will be pushed into drug container 10 and a valve (not shown) will be opened through which a defined amount of the drug is delivered. The functioning of such drug containers for delivering metered dose aerosols is known to a person skilled in the art.

In this exemplary embodiment, outlet 12 is stuck in channel 9, thus securing the inserted drug container 10 from falling out.

However, it is likewise conceivable for container housing 4 to be equipped with additional clamping means (not shown) which may in particular engage at collar 11. The atomizer outlet in the mouthpiece is substantially directed toward the opening of mouthpiece 2. Through this outlet whose exact configuration will be explained in more detail below, the aerosol cloud enters the mouthpiece.

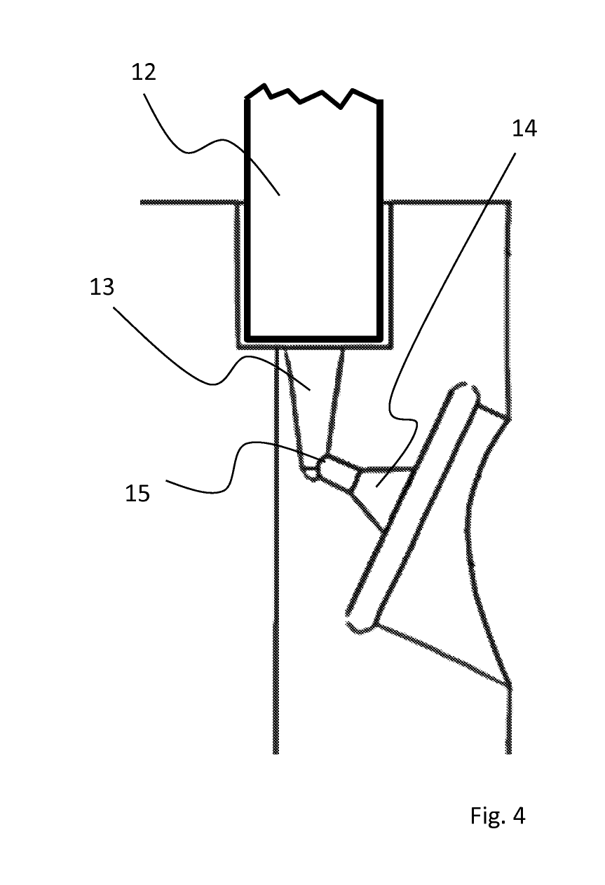

FIG. 4 is a detailed view of the channel between outlet 12 of the drug container and the atomizer outlet shown in FIG. 3.

The channel is divided into a first channel section 13 and a second channel section 14, which are arranged relative to each other at an angle from 110° to 120°. First and second channel sections 13, 14 are tapering and have a substantially truncated conical shape in this exemplary embodiment.

The two channel sections 13, 14 taken together have a length of less than 5 mm and a maximum diameter of less than 2 mm.

Between channel sections 13, 14, a constriction 15 is formed which in this exemplary embodiment has a substantially circular cylindrical shape in sections thereof and is arranged coaxially with channel section 14 and is thus arranged in correspondence to a central axis of the mouthpiece.

In this constriction which in the present exemplary embodiment has a diameter of less than 0.7 mm, the drug emitted from outlet 12 is considerably accelerated.

FIG. 4 only shows the plastic parts of the inhaler, which are integral parts of the housing which can be produced in one piece by injection molding.

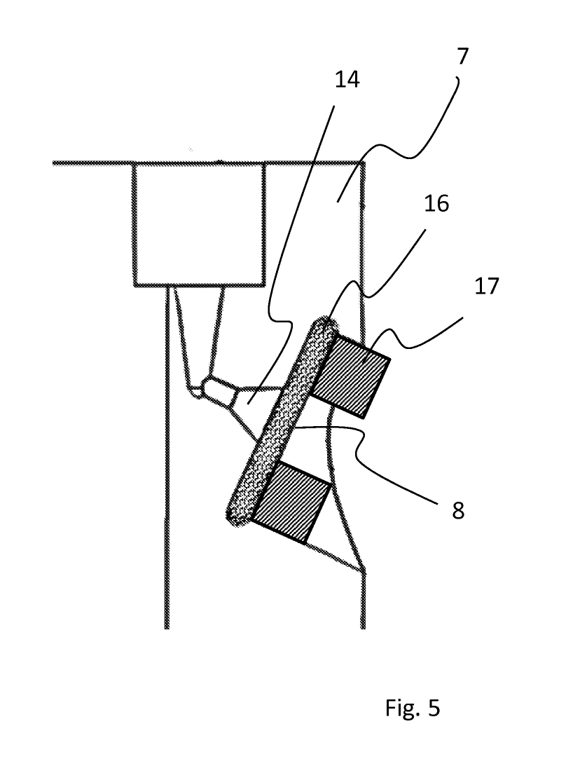

FIG. 5 is a view similar to FIG. 4 in which, now, a permeable sheet 16 is placed in front of channel section 14 and which serves as an atomizer outlet.

The permeable sheet may be formed as a mesh, in particular a nylon mesh having a pore diameter from 35 to 45 μm.

The mesh is secured by means of a ring 17 which may be fixed by gluing, welding, in particular cold welding, or clamping, for example. By means of ring 17 which is placed in a corresponding recess of seat 7, the permeable sheet 16 can be placed particularly easily.

In the context of the invention, the diameter of permeable sheet 16 refers to the diameter overlapping channel section 14. It will be understood that the portion of sheet 16 laterally thereof only serves for fastening purposes by means of ring 17, but is not defining an atomizer outlet.

The combination of a short channel with a constriction, which only has a small volume, and the placement of sheet 16 close to the outlet of the drug container provides for a pressure buildup in the channel, so that possibly a formation of droplets mainly occurs behind the atomizer outlet.

It has been found that in this manner the velocity of the particles emitted from the inhaler can be significantly reduced in a surprisingly simple way.

Measurements have shown that the velocity of the particles could be reduced by more than 40% compared to prior art inhalers available on the market.

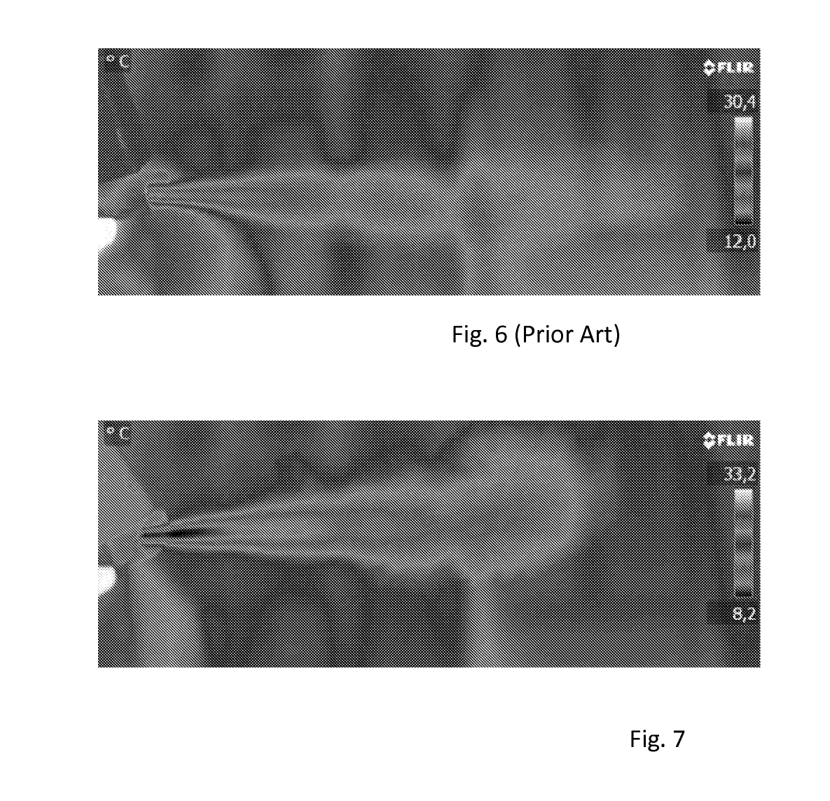

The effect of the invention will be illustrated with reference to the infrared images of FIG. 6 and FIG. 7.

FIG. 6 shows an infrared image of the aerosol cloud of an inhaler available on the market.

By contrast, FIG. 7 shows an infrared image of the aerosol cloud of an inhaler according to the invention. As can be seen, the aerosol cloud is substantially larger here. It has a volume of more than 150% of the volume of the aerosol cloud illustrated in FIG. 6.

Because of the lower particle velocity and the larger aerosol cloud, inhalation of the aerosol is considerably easier.

At the same time, the standard deviation of the delivered dose was even improved compared to known inhalers. In particular, a standard deviation of less than 10% was achieved.

At the same time it was achieved, that the emitted amount of the drug substantially corresponds to the emitted amount of the drug generated by an inhaler which only has a nozzle as the atomizer.

Experiments have shown that when the inhaler was used for a suspension, the aerosol cloud had a temperature increased by at least 2 degrees Celsius. In particular, temperatures increased by up to 4 degrees Celsius were achieved. The velocity of the aerosol particles was reduced by at least 20%, preferably up to a half. Simultaneously, an aerosol cloud with a volume of more than 1.5 times the volume, preferably up to twice the volume compared to that of a standard inhaler without a permeable sheet was generated.

When a solution was used, the number of respirable particles in the aerosol cloud was increased by up to ten percentage points (from 35% to 45%).

### LIST OF REFERENCE NUMERALS

1 Inhaler
2 Mouthpiece
3 Opening
4 Container housing
5 Opening
6 Extension
7 Seat
8 Atomizer outlet
9 Channel
10 Drug container
11 Collar
12 Outlet
13 Channel section
14 Channel section
15 Constriction
16 Permeable sheet
17 Ring

1. An inhaler for a metered dose aerosol, comprising:
a seat for an outlet of a drug container and a channel that extends from the seat to an atomizer outlet;
wherein the atomizer outlet comprises a permeable sheet, and wherein the channel is constricted in sections between the outlet of the drug container and the atomizer outlet.

2. The inhaler as claimed in claim 1, wherein the channel has at least two channel sections tapering toward a constriction.

3. The inhaler as claimed in claim 2, wherein the constriction has a diameter between 0.1 and 1 mm.

4. The inhaler as claimed in claim 1, wherein the channel is divided into two channel sections which are arranged relative to each other at an angle between 100° and 130°.

5. The inhaler as claimed in claim 1, wherein the permeable sheet is a mesh, a nonwoven fabric, or a woven fabric.

6. The inhaler as claimed in claim 1, wherein the permeable sheet has a mean pore diameter between 10 and 100 μm.

7. The inhaler as claimed in claim 1, wherein the permeable sheet has an open surface area between 10 and 60%.

8. An inhaler for a metered dose aerosol, comprising:
a seat for an outlet of a drug container and
a channel that extends from the seat to an atomizer outlet;
wherein the channel is closed by a permeable sheet and has a length of less than 8 mm.

9. The inhaler as claimed in claim 8, wherein the channel has a volume of less than 10 mm³.

10. An inhaler for a metered dose aerosol, comprising:
a container housing for receiving a drug container, and
an atomizer outlet which opens into a mouthpiece,
wherein the mouthpiece has an opening and an extension at an end opposite the opening.

11. The inhaler as claimed in claim 2, wherein the constriction has a diameter between 0.3 and 0.7 mm.

12. The inhaler as claimed in claim 2, wherein the constriction has a diameter between 0.2 and 0.3 mm.

13. The inhaler as claimed in claim 2, wherein the constriction has a diameter between 0.4 and 0.6 mm.

14. The inhaler as claimed in claim 1, wherein the channel is divided into two channel sections which are arranged relative to each other at an angle between 110° and 120°.

15. The inhaler as claimed in claim 1, wherein the permeable sheet has a mean pore diameter between 30 and 50 μm.

16. The inhaler as claimed in claim 1, wherein the permeable sheet has an open surface area between 25 and 45%.

17. The inhaler as claimed in claim 8, wherein the channel has a volume of less than 5 mm³.

18. The inhaler as claimed in claim 8, wherein the channel has a volume of less than 3 mm³.
